(19) United States
(12) Patent Application Publication
(10) Pub. No.: US 2017/0210793 A1
(43) Pub. Date: Jul. 27, 2017

(54) Novel Treatment for Polycystic Kidney Disease

(71) Applicant: The Regents of the University of California, Oakland, CA (US)

(72) Inventor: THOMAS WEIMBS, Goleta, CA (US)

(21) Appl. No.: 15/326,296

(22) PCT Filed: Jul. 14, 2015
(86) PCT No.: PCT/US15/40410

§ 371 Date: Jan. 13, 2017

Related U.S. Application Data
(60) Provisional application No. 62/024,748, filed on Jul. 15, 2014.

(51) Int. Cl. C07K 16/22; C07K 16/32; C07K 16/28; C07K 16/24

(52) U.S. Cl. CPC C07K 16/22; C07K 16/244; C07K 16/32; C07K 16/2863; C07K 16/247; C07K 2317/76; A61K 2039/505

(57) ABSTRACT

The invention comprises the administration of dimeric IgA or pentameric IgM antibodies to animals, including human patients, suffering from a disease state wherein the polymeric immunoglobulin receptor is expressed, such antibodies comprising antibodies that will neutralize one or more growth factors associated with the disease state, or their receptors, in order to diminish the onset, progression, and growth of diseased tissues. The polymeric immunoglobulin receptor is expressed in diseased tissues such as in the apical membranes of cyst-lining cells in polycystic kidney disease.

### CROSS-REFERENCE TO RELATED APPLICATIONS:

This application claims the benefit of priority to U.S. Provisional Application Ser. No. 62/024,748, entitled “Novel Treatments for Polycystic Kidney Disease,” filed Jul. 15, 2014, the contents which are hereby incorporated by reference.

### STATEMENT REGARDING FEDERALLY SPONSORED RESEARCH OR DEVELOPMENT

This invention was made with government support under grant number R01 DK62338 awarded by the National Institutes of Health. The government has certain rights in the invention.

### BACKGROUND OF THE INVENTION

Autosomal dominant polycystic kidney disease (ADPKD) is considered the most common life-threatening monogenic disease. The ADPKD disease state is primarily characterized by the growth of fluid-filled cysts in the kidneys which progressively enlarge, leading to destruction of normal renal tissue and function. Renal cyst growth in ADPKD is driven by several growth factors, hormones and cytokines that are present in the cyst fluid and which stimulate the cyst-lining epithelial cells. There is currently no FDA-approved treatment to prevent the progression of ADPKD, which affects more than 12.5 million people worldwide and 600,000 in the United States.

### SUMMARY OF THE INVENTION

Disclosed herein are novel compositions and methods for the treatment of ADPKD and like disorders. The inventors of the present disclosure have advantageously discovered that the cysts in ADPKD patients express high levels of the polymeric immunoglobulin receptor (pIgR), which transports certain polymeric immunoglobulins, primarily dimeric immunoglobulin A (dIgA) or pentameric immunoglobulin M (pIgM) antibodies, into the cyst lumen. The invention comprises the targeting to polycystic renal tissues of such dimeric IgA or pentameric IgM antibodies, wherein the antibodies specifically bind and inhibit growth factors (or their receptors) or other molecular targets which are involved in cyst growth. The administration of such antibodies inhibits such ADPKD-associated molecular targets and thereby attenuate the condition by interfering with growth factor mediated processes which drive cyst formation, growth, and progression.

Compared to any prior art, there are two main advantages of the pIgR-mediated targeting of therapeutic antibodies to renal cysts in ADPKD patients. First, pIgR-mediated targeting enables therapeutic antibodies in dIgA or pIgM format to gain access to renal cyst lumens and the apical membranes of cyst-lining cells. Second, pIgR-mediated enrichment of therapeutic antibodies in dIgA or pIgM format in renal cysts provides a means of reducing extra-renal side effects.

In one aspect, the invention comprises a pharmaceutical composition comprising a dimeric IgA or pentameric IgM antibody which neutralizes an ADPKD-associated molecular target. In another aspect, the invention comprises a method of treating ADPKD with such antibodies. In another aspect, the invention comprises methods of using dimeric IgA or pentameric IgM antibodies to treat other conditions wherein expression of pIgR will facilitate targeting and enrichment of these antibodies to the diseased target tissue compartment.

### BRIEF DESCRIPTION OF THE DRAWINGS

FIG. 1. Accumulation of human dIgA in polycystic kidneys compared to wild type kidneys when analyzed 24 hours post-injection. Detection was achieved using an antibody specific to human kappa light chain. n=2, mean±range.

FIG. 2. IL4 and IL13 induce expression of pIgR in kidney epithelial cells. Treatment of IMCD cells with 100 ng/mL mouse IL4 or mouse IL13 increases the expression of pIgR mRNA when analyzed by qPCR (*p+<0.05, two-tailed t-test, representative graph from three independent experiments).

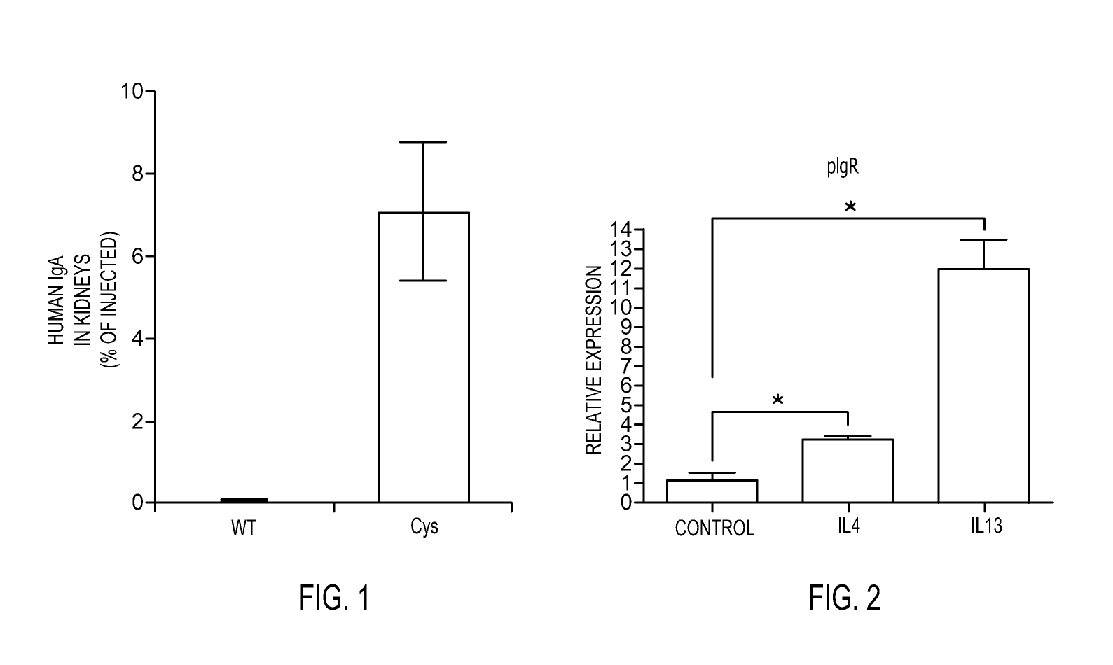

### DETAILED DESCRIPTION OF THE INVENTION

ANTIBODIES. In one aspect, the invention encompasses a dimeric IgA or a pentameric IgM antibody which will neutralize or inhibit an ADPKD-associated molecular target. As used herein, an “ADPKD-associated molecular target” is any growth factor, hormone, cytokine, growth factor receptor, hormone receptor, cytokine receptor or other species which is implicated in ADPKD cyst formation, growth, or persistence. For example, a list follows of molecular targets that have been identified as aberrantly activated in renal cysts. These growth factors, and their receptors, are deemed to be ADPKD-associated molecular targets: interleukin-13 (IL13); interleukin-4 (IL4); the IL4/13 receptor; epidermal growth factor (EGF) and its receptor; hepatocyte growth factor (HGF) and its receptor c-Met; transforming growth factor alpha (TGF-alpha) and its receptor; tumor necrosis factor alpha (TNF-alpha) and its receptor; interleukin 6 (IL-6) and its receptor; transforming growth factor-β (TGF-beta) and its receptor; HER2; platelet-derived growth factor (PDGF) and its receptor; and ouabain and its receptor. An ADPKD-associated molecular target, as used herein, further includes receptors, precursors, effectors, and downstream species regulated by the ADPKD-associated growth factors enumerated above or any other growth factors found to be involved in cyst formation, growth, and persistence. It will be understood that the molecular targets enumerated herein are human growth factors and receptors, however, antibodies to the homologous or orthologous targets in non-human species are within the scope of the invention as well.

A “neutralizing” antibody is one that inhibits one or more signaling actions of a molecular target, for example by interfering with ligand-receptor interactions or otherwise reducing or ablating the action of the target species. Neutralization of the target moiety by the neutralizing antibody is accomplished by selective binding to one or more epitopes of the molecular target. Neutralization includes any measurable reduction in activity, including total obliteration of a signaling activity.

An “IgA antibody,” as used herein, means an antibody having immunoglobulin light chains in combination with the heavy chains containing constant regions comprising known human IgA sequences. Exemplary IgA sequences include, Ig alpha-1 chain C region, NCBI ACCESSION P01876 or Ig alpha-2 chain C region, NCBI ACCESSION P01877, and variants thereof, as well as sequences described in U.S. Pat. No. 8,236,561 by Jones, entitled “Efficient Production of IgA in Recombinant Mammalian Cells Humanized or chimeric antibodies comprising substantial (e.g. greater than 10%) IgA sequence identity are also within the scope of the invention. Hybrid antibodies, comprising IgA and non-IgA regions, are within the scope of the invention, to the extent such hybrid antibodies, when dimerized, will be trancytosed by the polymeric immunoglobulin receptor.

The IgA antibodies of the invention further comprise a variable region in each of the light and heavy chains, these variable regions having complementarity determining regions and framework regions, as known in the art. The IgA antibodies of the invention are further defined by functionality, in that one or more elements of their variable region will specifically bind to an epitope within a molecular target. The use of bispecific IgA antibodies, wherein each arm of the antibody is specific for a different antigen, wherein at least one antigen is an epitope of a molecular target, is within the scope of the invention as well. IgA antibodies to molecular targets may comprise recombinantly produced antibodies or antibodies formed in hybridomas or other cellular sources, including IgA antibodies produced in transgenic animals, plants, or cell cultures. The variable regions of the IgA antibodies of the invention may comprise antigen-binding regions derived from non-IgA antibodies, such as IgG or non-human antibodies developed against a molecular target. The scope of the invention encompasses both IgA1 and IgA2 antibodies. The scope of the invention further encompasses glycosylated IgA antibodies.

A polymeric IgA antibody is two or more monomeric IgA antibodies bound together as a dimer or higher polymer by the human J chain (for example, the sequence described as NCBI Reference Sequence: NP_653247.1). In one implementation of the invention, the IgA antibodies are dimerized so as to be competent for transport by the human polymeric immunoglobulin receptor. Dimeric IgA antibodies are joined by a peptide known as the J-chain. Methods of dimerizing of IgA antibodies are known in the art, for example as described in U.S. Pat. No. 8,021,645, by Simon et al., entitled “Synthesis of human secretory IgA and IgM and the formulation of a medicament therefrom”; U.S. Pat. No. 6,063,905 by Capra et al., entitled “Recombinant IgJ Chain Dimer”; United States Patent Number; and in United States Patent Application Publication Number 20140371431, by Brown et al., entitled “Process for preparation of secretory IgA and secretory IgM.” In one aspect, the dimerized IgA antibodies of the invention comprise known monomeric IgA antibodies which are dimerized utilizing methods known in the art. In one embodiment, the dimerized IgA antibodies of the invention are homodimers and in another embodiment the IgA antibodies of the invention are heterodimers.

Although the description herein is primarily directed to dimeric IgA antibodies for therapeutic uses, it will be understood that higher order (e.g. trimer, tetramer, pentamer, etc.) combinations of IgA antibodies and their therapeutic applications are within the scope of the invention as well.

An “IgM antibody,” as used herein means an antibody having immunoglobulin light chains in combination with the heavy chains containing constant regions comprising known human IgM sequences. Exemplary IgM sequences include the Ig mu chain C region, NCBI ACCESSION P01871 and variants thereof, as well as sequences described in Tchoudakova et al., “High level expression of functional human IgMs in human PER.C6 cells,” MAbs. 2009 March-April; 1(2): 163-171; United States Patent Application Publication Number 20060063234, by Jones, entitled “Efficient production of igM in recombinant mammalian cells”; PCT Application Publication Number WO2014083006, by Katinger et al., entitled “Recombinant human IgM antibody effective against cancer cells.” Humanized or chimeric antibodies comprising substantial (e.g. greater than 10%) IgM sequence identity are also within the scope of the invention. Hybrid antibodies, comprising IgM and non-IgM regions, are within the scope of the invention, to the extent such hybrid antibodies, when pentameric, will be trancytosed by the polymeric immunoglobulin receptor. For veterinary treatment, the equivalent orthologous immunoglobulin and J-chain species are also within the scope of the invention.

The IgM antibodies of the invention further comprise a variable region in each of the light and heavy chains, these variable regions having complementarity determining regions and framework regions, as known in the art. The IgM antibodies of the invention are further defined by functionality, in that one or more elements of their variable region will specifically bind to a molecular target. The use of bispecific IgM antibodies, wherein each arm of the antibody is specific for a different antigen, wherein at least one antigen is an epitope of a molecular target, is within the scope of the invention. IgM antibodies to molecular targets may comprise recombinantly produced antibodies or antibodies formed in hybridomas or other cellular sources, including IgM antibodies produced in transgenic animals, plants, or cell cultures. The variable regions of the IgM antibodies of the invention may comprise antigen-binding regions derived from non-IgM antibodies, such as IgG antibodies developed against a molecular target. The scope of the invention further encompasses glycosylated IgM antibodies.

A polymeric IgM antibody is two or more monomeric IgM antibodies bound together as a dimer or higher polymer by the human J chain (for example, the sequence described as NCBI Reference Sequence: NP_653247.1). In one implementation of the invention, the IgM antibodies are pentameric so as to be competent for transport by the human polymeric immunoglobulin receptor. Pentameric IgM antibodies are joined by a peptide known as the J-chain. Methods of pentamerizing of IgM antibodies are known in the art, for example as described in U.S. Pat. No. 8,021,645, by Simon et al., entitled “Synthesis of human secretory IgA and IgM and the formulation of a medicament therefrom”; U.S. Pat. No. 6,063,905 by Capra et al., entitled “Recombinant IgJ Chain Dimer”; United States Patent Number; and in United States Patent Application Publication Number 20140371431, by Brown et al., entitled “Process for preparation of secretory IgA and secretory IgM.” In one aspect, the pentameric IgM antibodies of the invention comprise known monomeric IgM antibodies which are pentameric utilizing methods known in the art. In one embodiment, the pentameric IgM antibodies of the invention comprise a single type of antibody and in another embodiment the pentameric IgM antibodies of the invention are heterogeneous, comprising IgM antibodies having specificity for different molecular targets.

Although the description herein is primarily directed to pentameric IgM antibodies for therapeutic uses, it will be understood that lower order (e.g. dimer, trimer, tetramer, etc.) and higher order (e.g. hexamer) combinations of IgM antibodies and their therapeutic applications are within the scope of the invention as well.

In one aspect, the invention is directed to the treatment of a condition in which the affected target tissue or organ expresses the pIgR. Expression of pIgR in the diseased tissue provides a means of selectively targeting the antibodies of the invention to the luminal spaces of such diseased tissue. The pIgR will trancytose dimeric IgA or pentameric IgM antibodies into the cells or luminal structures (e.g. cysts) of the diseased tissue. The scope of the invention includes dimeric IgA antibodies and pentameric IgM antibodies having variable regions which neutralize molecular targets present in a diseased tissue, such diseased tissue being associated with expression of the pIgR.

In the case of ADPKD, expression of pIgR in renal cyst cells provides a means of selectively targeting the antibodies of the invention to renal cysts. The pIgR will trancytose dimeric IgA or pentameric IgM antibodies into the lumen of the cyst. The scope of the invention includes dimeric IgA antibodies and pentameric IgM antibodies having variable regions which bind and neutralize one or more ADPKD-associated molecular targets. The scope of the invention further encompasses a method of treating ADPKD by the administration of such antibodies.

Molecular targets may comprise growth factors associated with a particular disease, such as ADPKD. Molecular targets may further comprise receptors for growth factors associated with a particular disease state. It will be understood that many growth factor ligands have multiple receptors, and the antibodies of the invention directed to a receptor of a specific growth factor encompass those that will specifically bind to any receptor for the enumerated growth factor.

In one embodiment, the invention encompasses the use of a dimeric IgA or pentameric IgM antibody which neutralizes the IL4 growth factor in the treatment of ADPKD or other pIgR-associated conditions. In another embodiment, the invention comprises a dimeric IgA or pentameric IgM antibody which neutralizes the IL4 growth factor. In another embodiment, the invention comprises a dimeric IgA or a pentameric IgM antibody which neutralizes the IL4 growth factor for use in the treatment of ADPKD or other pIgR-associated conditions. For example, the antigen-binding domain of such IL4-neutralizing antibodies may comprise an IL4 binding domain disclosed in U.S. Pat. No. 8,388,965, by Rao et al., entitled “Antibodies that bind to IL4 or IL13 and their uses” and in United States Patent Application Publication Number 20100297110, by Hoeger et al., entitled “Antibody specific for it-4 for the treatment of cancer.” Anti IL4 antibodies comprising IgA or IgM constant domains are contemplated in the prior art, however, to the knowledge of the inventor of the present disclosure, dimerized IgA and pentameric IgM antibodies to IL4 have not previously been disclosed.

In one embodiment, the invention encompasses the use of an IgA or IgM antibody having an antigen-binding domain which neutralizes a receptor of IL4 in the treatment of ADPKD or other pIgR-associated conditions. In another embodiment, the invention comprises a dimeric IgA or a pentameric IgM antibody which neutralizes an IL4 receptor. In another embodiment, the invention comprises a dimeric IgA or a pentameric IgM antibody which neutralizes an IL4 receptor for use in the treatment of ADPKD or other pIgR-associated conditions. For example, the antigen-binding region of such IL4 receptor neutralizing antibodies may comprise an antigen-binding domain disclosed in U.S. Pat. No. 8,679,497, by Armitage, entitled “Anti interleukin-4 receptor antibodies”; U.S. Pat. No. 8,337,839, by Martin, entitled “High Affinity Human Antibodies to human IL-4 Receptor”; U.S. Pat. No. 8,337,839, by Martin, entitled “High Affinity Human Antibodies to human IL-4 Receptor.” Monomeric anti-IL4 receptor antibodies comprising IgA or IgM heavy and light chain constant regions are contemplated in the prior art, however, dimerized IgA and pentameric IgM against an IL4 receptor, to the knowledge of the inventor of the present disclosure, have not been previously disclosed.

In one embodiment, the invention comprises a dimeric IgA or a pentameric IgM antibody having an antigen-binding domain which neutralizes the IL13 growth factor in the treatment of ADPKD or other pIgR-associated conditions. In another embodiment, the invention comprises a dimeric IgA or a pentameric IgM antibody which neutralizes the IL13 growth factor. In another embodiment, the invention comprises a dimeric IgA or a pentameric IgM antibody which neutralizes the IL13 growth factor for use in the treatment of ADPKD or other pIgR-associated conditions. For example, the antigen-binding domain of such IL13 antibodies may comprise an IL13 binding domain disclosed in U.S. Pat. No. 8,388,965, by Rao et al., entitled “Antibodies that bind to IL4 or IL13 and their uses”; United States Patent Application Publication Number 20090060906, by Barry et al., entitled “Anti-Il-13 antibody formulations and uses thereof”; U.S. Pat. No. 8,734,801, by Fung et al., entitled “Anti-II13 antibodies and uses thereof”; and U.S. Pat. No. 8,399,630, by Swanson, entitled “Engineered anti-Il13 antibodies, compositions, methods, and uses.” Monomeric antibodies against IL13 comprising IgA or IgM constant domains are contemplated in the prior art, however, to the knowledge of the inventor of the present disclosure, dimerized IgA and pentameric IgM antibodies against IL13 have not been previously disclosed.

In one embodiment, the invention comprises a dimeric IgA or a pentameric IgM antibody having an antigen-binding domain which neutralizes a receptor of IL13 in the treatment of ADPKD or other pIgR-associated conditions. In another embodiment, the invention comprises a dimeric IgA or a pentameric IgM antibody which neutralizes an IL13 receptor. In another embodiment, the invention comprises a dimeric IgA or a pentameric IgM antibody which neutralizes an IL13 receptor for use in the treatment of ADPKD or other pIgR-associated conditions. For example, the antigen-binding domain of such IL13 receptor neutralizing antibodies may comprise a binding domain disclosed in U.S. Pat. No. 7,807,158, by Endl, entitled “Antibodies against IL-13 receptor alpha one and uses thereof”; PCT Patent Application Publication Number 2014072888, by Ma et al., entitled “Anti IL-13 receptor alpha two antibodies and antibody drug conjugates”; and Krause, et al., “Blockade of interleukin-13-mediated cell activation by a novel inhibitory antibody to human IL-13 receptor_1,” Mol. Immunol. 43, 1799 (2006). Monomeric anti-IL13 receptor antibodies comprising IgA and IgM constant domains are contemplated in the prior art, however, to the knowledge of the inventor of the present disclosure, dimerized IgA and pentameric IgM antibodies to an IL13 receptor have not been previously disclosed.

In one embodiment, the invention encompasses the use of a dimeric IgA or a pentameric IgM antibody which neutralizes epidermal growth factor (EGF) in the treatment of ADPKD or other pIgR-associated conditions. In another embodiment, the invention comprises a dimeric IgA antibody or a pentameric IgM antibody which neutralizes EGF. In another embodiment, the invention comprises a dimeric IgA antibody or a pentameric IgM antibody which neutralizes EGF for use in the treatment of ADPKD or other pIgR-associated conditions. For example, the antigen-binding domain of such EGF antibodies may comprise a binding domain disclosed in United States Patent Application Publication Number 20100273988, by Kimura, entitled “Anti-cancer agent comprising anti-HB-EGF antibody as active ingredient.” Monomeric anti-EGF antibodies comprising IgA and IgM constant domains are contemplated in the prior art, however, to the knowledge of the inventor of the present disclosure, dimerized IgA and pentameric IgM antibodies to EGF have not been previously disclosed.

In one embodiment, the invention encompasses the use of a dimeric IgA or a pentameric IgM antibody which neutralizes a receptor of EGF in the treatment of ADPKD or other pIgR-associated conditions. In another embodiment, the invention comprises a pentameric IgM antibody which neutralizes a receptor EGF. In another embodiment, the invention comprises a dimeric IgA or a pentameric IgM antibody which neutralizes a receptor EGF for use in the treatment of ADPKD or other pIgR-associated conditions. For example, the antigen-binding domain of such EGF receptor neutralizing antibodies may comprise a binding domain disclosed in United States Patent Application Publication Number 20120344093 by Daly et al., entitled “Anti-EGFR antibodies and uses thereof;” United States Patent Application Publication Number 20110177068, by Mueller and Mahler, entitled “Pharmaceutical composition comprising an antibody against the EGF receptor” and in United States Patent Application Publication Number 20110150759, by Johns et al., entitled “Monoclonal antibody 175 targeting the EGF receptor and derivatives and uses thereof.” Monomeric anti-EGFR antibodies comprising IgM constant domains are contemplated in the prior art, however, to the knowledge of the inventor of the present disclosure, pentameric IgM antibodies to an EGFR have not been previously disclosed.

In one embodiment, the invention encompasses the use of a dimeric IgA or a pentameric IgM antibody which neutralizes HER2 (erbB-2 or Neu) in the treatment of ADPKD or other pIgR-associated conditions. In another embodiment, the invention comprises a dimeric IgA antibody or a pentameric IgM antibody which neutralizes HER2. In another embodiment, the invention comprises a dimeric IgA antibody or a pentameric IgM antibody which neutralizes HER2 for use in the treatment of ADPKD or other pIgR-associated conditions. For example, the antigen-binding domain of such HER2 antibodies may comprise the binding domain of Herceptin™ (Genentech), as known in the art. Further HER2-binding domains are described in U.S. Pat. No. 7,560,111, by Kao and Vanderlann, entitled “Her2 Antibody Composition”; PCT Patent Application Publication Number WO2011130580, by Alper, entitled “Monoclonal antibodies against Her2 antigens and uses therefor”; and U.S. Pat. No. 5,677,171, by Hudziak et al., entitled “Monoclonal antibodies directed to the Her2 receptor.” Monomeric anti-HER2 antibodies comprising IgA or IgM constant regions are contemplated in the prior art, however, to the knowledge of the inventor of the present disclosure, dimeric IgA or pentameric IgM antibodies which neutralize HER2 have not previously been disclosed.

In one embodiment, the invention encompasses the use of a dimerc IgA or a pentameric IgM antibody which neutralizes hepatocyte growth factor (HGF) in the treatment of ADPKD or other pIgR-associated conditions. In another embodiment, the invention comprises a dimeric IgA antibody or a pentameric IgM antibody which neutralizes HGF. In another embodiment, the invention comprises a dimeric IgA antibody or a pentameric IgM antibody which neutralizes HGF for use in the treatment of ADPKD or other pIgR-associated conditions. For example, the antigen-binding domain of such HGF-neutralizing antibodies may comprise a binding domain disclosed in United States Patent Application Publication Number 20090023894, by Junho and Youngmi, entitled “Neutralizing Antibody Against HGF” and U.S. Pat. No. 7,459,536 by Cao and Woude, entitled “HGF-SF Monoclonal Antibody Combinations”; and United States Patent Application Publication Number 20140271459, by Dutzar et al., entitled “Antibodies to hgf and compositions containing.” Monomeric anti-HGF antibodies comprising IgA or IgM constant regions are contemplated in the prior art, however, to the knowledge of the inventor of the present disclosure, dimeric IgA or pentameric IgM antibodies which neutralize HGF have not previously been disclosed.

In one embodiment, the invention encompasses the use of a dimeric IgA or a pentameric IgM antibody which neutralizes the receptor for hepatocyte growth factor (HGFR or c-Met) in the treatment of ADPKD or other pIgR-associated conditions. In another embodiment, the invention comprises a dimeric IgA antibody or a pentameric IgM antibody which neutralizes c-Met. In another embodiment, the invention comprises a dimeric IgA antibody or a pentameric IgM antibody which neutralizes c-Met for use in the treatment of ADPKD or other pIgR-associated conditions. For example, the antigen-binding domain of such c-Met-neutralizing antibodies may comprise a binding domain disclosed in U.S. Patent Application Publication Number 20130315895, by Farrel and Vincent, entitled “Combination of a c-Met antibody and an antibody to HGF and/or c-Met”; United States Patent Application Publication Number 20140193431, by Park et al., entitled “Anti c-Met Antibody Having HGF Activity and Uses Thereof”; and United States Patent Application Publication Number 20140294814, by Lee et al., entitled “Humanized and affinity matured anti c-met antibody and uses thereof.” Monomeric anti-HGF antibodies comprising IgA or IgM constant regions are contemplated in the prior art, however, to the knowledge of the inventor of the present disclosure, dimeric IgA or pentameric IgM antibodies which neutralize HGF have not previously been disclosed.

In one embodiment, the invention comprises the use of a dimeric IgA or a pentameric IgM antibody having an antigen-binding domain which neutralizes ouabain in the treatment of ADPKD or another pIgR-associated condition. In one embodiment, the invention comprises a dimeric IgA or a IgM antibody which neutralizes ouabain. In one embodiment, the invention comprises a dimeric IgA or a pentameric IgM antibody which neutralizes ouabain for the treatment of ADPKD or another pIgR-associated condition. For example, the antigen-binding domain of such ouabain neutralizing antibodies may comprise a binding domains disclosed in U.S. Pat. No. 5,429,928, by Blaustein et al., entitled “Immunoassay for detecting human ouabain.”

In one embodiment, the invention comprises the use of a dimeric IgA or a pentameric IgM antibody having an antigen-binding domain which neutralizes a receptor of ouabain in the treatment of ADPKD or another pIgR-associated condition. In one embodiment, the invention comprises a dimeric IgA or a IgM antibody which neutralizes a receptor of ouabain. In one embodiment, the invention comprises a dimeric IgA or a pentameric IgM antibody which neutralizes a receptor of ouabain for the treatment of ADPKD or another pIgR-associated condition.

In one embodiment, the invention encompasses the use of a dimerc IgA or a pentameric IgM antibody which neutralizes transforming growth factor alpha (TGF-α) in the treatment of ADPKD or other pIgR-associated conditions. In another embodiment, the invention comprises a dimeric IgA antibody or a pentameric IgM antibody which neutralizes TGF-α. In another embodiment, the invention comprises a dimeric IgA antibody or a pentameric IgM antibody which neutralizes TGF-α for use in the treatment of ADPKD or other pIgR-associated conditions. For example, the antigen-binding domain of such TGF-α-neutralizing antibodies may comprise a binding domain disclosed in United States Patent Application Publication Number 20130131322, by Kaneda et al., entitled “Antibody being capable of binding to transforming growth factor alpha and having growth-suppressing on cancers having RAS gene mutation.”

In one embodiment, the invention encompasses the use of a dimerc IgA or a pentameric IgM antibody which neutralizes a receptor of transforming growth factor alpha (TGF-α) in the treatment of ADPKD or other pIgR-associated conditions. In another embodiment, the invention comprises a dimeric IgA antibody or a pentameric IgM antibody which neutralizes a receptor of TGF-a. In another embodiment, the invention comprises a dimeric IgA antibody or a pentameric IgM antibody which neutralizes a receptor of TGF-a for use in the treatment of ADPKD or other pIgR-associated conditions.

In one embodiment, the invention encompasses the use of a dimeric IgA or a pentameric IgM antibody which neutralizes transforming growth factor beta (TGF-β) in the treatment of ADPKD or other pIgR-associated conditions. In another embodiment, the invention comprises a dimeric IgA antibody or a pentameric IgM antibody which neutralizes TGF-β. In another embodiment, the invention comprises a dimeric IgA antibody or a pentameric IgM antibody which neutralizes TGF-β for use in the treatment of ADPKD or other pIgR-associated conditions. For example, the antigen-binding domain of such TGF-β-neutralizing antibodies may comprise a binding domain disclosed in United States Patent Application Publication Number 20080267946, by Kim et al., entitled “Pharmaceutical Composition for Treating Avellino Cornea Dystrophy Comprising an Antibody Against Tgf-Beta”; United States Patent Application Publication Number 20050276802, by Adams et al., entitled “Humanized anti-TGF-beta antibodies”; and United States Patent Application Publication Number 20110008364, by Ledbetter et al., entitled “Antibodies to tgf-beta.” Monomeric anti-TGF-βantibodies comprising IgA or IgM constant regions are contemplated in the prior art, however, to the knowledge of the inventor of the present disclosure, dimeric IgA or pentameric IgM antibodies which neutralize TGF-β have not previously been disclosed.

In one embodiment, the invention encompasses the use of a dimeric IgA or a pentameric IgM antibody which neutralizes a receptor of transforming growth factor beta (TGF-β) in the treatment of ADPKD or other pIgR-associated conditions. In another embodiment, the invention comprises a dimeric IgA antibody or a pentameric IgM antibody which neutralizes a receptor of TGF-β. In another embodiment, the invention comprises a dimeric IgA antibody or a pentameric IgM antibody which neutralizes a receptor of TGF-β for use in the treatment of ADPKD or other pIgR-associated conditions. For example, the antigen-binding domain of such TGF-β receptor-neutralizing antibodies may comprise a binding domain disclosed in U.S. Pat. No. 8,147,834, entitled “Anti TGF-beta receptor II antibodies”; U.S. Pat. No. 7,579,186, by Sakamoto et al., entitled “Human monoclonal antibody against TGF-β type II receptor and medicinal uses thereof. Monomeric anti-TGF-β receptor antibodies comprising IgA or IgM constant regions are contemplated in the prior art, however, to the knowledge of the inventor of the present disclosure, dimeric IgA or pentameric IgM antibodies which neutralize TGF-β receptors have not previously been disclosed.

In one embodiment, the invention encompasses the use of a dimeric IgA or a pentameric IgM antibody which neutralizes inteleukin-6 (IL6) in the treatment of ADPKD or other pIgR-associated conditions. In another embodiment, the invention comprises a dimeric IgA antibody or a pentameric IgM antibody which neutralizes IL6. In another embodiment, the invention comprises a dimeric IgA antibody or a pentameric IgM antibody which neutralizes IL6 for use in the treatment of ADPKD or other pIgR-associated conditions. For example, the antigen-binding domain of such IL6-neutralizing antibodies may comprise a binding domain disclosed in U.S. Pat. No. 8,536,308, by Way, entitled “Antibodies to interleukin-6” and in U.S. Pat. No. 8,075889 by Gelinas et al., entitled “Antibody molecules having specificity for human il-6”; and in United States Patent Application Publication Number 20140112935, by Lin et al., entitled “Antibodies interleukin-6 and uses thereof.” Monomeric anti-IL6 antibodies comprising IgA or IgM constant regions are contemplated in the prior art, however, to the knowledge of the inventor of the present disclosure, dimeric IgA or pentameric IgM antibodies which neutralize IL6 have not previously been disclosed.

In one embodiment, the invention encompasses the use of a dimeric IgA or a pentameric IgM antibody which neutralizes a receptor of inteleukin-6 (IL6) in the treatment of ADPKD or other pIgR-associated conditions. In another embodiment, the invention comprises a dimeric IgA antibody or a pentameric IgM antibody which neutralizes a receptor of IL6. In another embodiment, the invention comprises a dimeric IgA antibody or a pentameric IgM antibody which neutralizes a receptor of IL6 for use in the treatment of ADPKD or other pIgR-associated conditions. For example, the antigen-binding domain of such IL6 receptor-neutralizing antibodies may comprise a binding domain disclosed in U.S. Pat. No. 8,043,617, by Stevens et al., entitled “Human antibodies to human IL-6 receptor”; U.S. Pat. No. 7,582,298 by Stevens et al., entitled “High affinity antibodies to human IL-6 receptor”; and in U.S. Patent Number 8,562,991, by Igawa et al., entitled “Antibodies that bind to IL-6 receptor.” Monomeric anti-IL6 receptor antibodies comprising IgA or IgM constant regions are contemplated in the prior art, however, to the knowledge of the inventor of the present disclosure, dimeric IgA or pentameric IgM antibodies which neutralize IL6 receptors have not previously been disclosed.

In one embodiment, the invention encompasses the use of a dimeric IgA or a pentameric IgM antibody which neutralizes tumor necrosis factor-alpha (TNF-α) in the treatment of ADPKD or other pIgR-associated conditions. In another embodiment, the invention comprises a dimeric IgA antibody or a pentameric IgM antibody which neutralizes TNF-α. In another embodiment, the invention comprises a dimeric IgA antibody or a pentameric IgM antibody which neutralizes TNF-α for use in the treatment of ADPKD or other pIgR-associated conditions. For example, the antigen-binding domain of such TNF-α-neutralizing antibodies may comprise a binding domain of a therapeutic antibody such as infliximab, adalimumab, golimumab, as known in the art or a binding domain disclosed in United States Patent Application Publication Number 20140186434, by Smith and Smith, entitled “Anti-tumor necrosis factor alpha (TNF-A) antibody used as a targeting to treat arthritis and other diseases”; and in United States Patent Application Publication Number 20140086904 by Ke and Gao, entitled “Humanized Anti-TNF-alpha Antibody and Antigen-Binding Fragment (Fab) Thereof and Use of the Same”. Monomeric anti-TNF-α antibodies comprising IgA or IgM constant regions are contemplated in the prior art, however, to the knowledge of the inventor of the present disclosure, dimeric IgA or pentameric IgM antibodies which neutralize TNF-α have not previously been disclosed.

In one embodiment, the invention encompasses the use of a dimeric IgA or a pentameric IgM antibody which neutralizes a receptor of tumor necrosis factor-alpha (TNF-α) in the treatment of ADPKD or other pIgR-associated conditions. In another embodiment, the invention comprises a dimeric IgA antibody or a pentameric IgM antibody which neutralizes a receptor of TNF-α. In another embodiment, the invention comprises a dimeric IgA antibody or a pentameric IgM antibody which neutralizes a receptor of TNF-α for use in the treatment of ADPKD or other pIgR-associated conditions. For example, the antigen-binding domain of such TNF-α receptor-neutralizing antibodies may comprise a binding domain disclosed in U.S. Pat. No. 7,728,111, by Wallach, entitled “Anti-p55 TNF alpha receptor antibodies”; and U.S. Pat. No. 6,262,239, by Wallach, entitled “TNF receptor specific antibodies.” Monomeric anti-TNF-α receptor antibodies comprising IgA or IgM constant regions are contemplated in the prior art, however, to the knowledge of the inventor of the present disclosure, dimeric IgA or pentameric IgM antibodies which neutralize a receptor of TNF-α have not previously been disclosed.

In one embodiment, the invention encompasses the use of a dimeric IgA or a pentameric IgM antibody which neutralizes platelet derived growth factor (PDGF), including the alpha, beta, other isoforms thereof, in the treatment of ADPKD or other pIgR-associated conditions. In another embodiment, the invention comprises a dimeric IgA antibody or a pentameric IgM antibody which neutralizes PDGF. In another embodiment, the invention comprises a dimeric IgA antibody or a pentameric IgM antibody which neutralizes PDGF for use in the treatment of ADPKD or other pIgR-associated conditions. For example, the antigen-binding domain of such PDGF-neutralizing antibodies may comprise a binding domain disclosed in PCT Patent Application Publication Number WO2013160359, by Fromond et al., entitled “Anti PDGF-c antibodies”; PCT Patent Application Publication Number WO2014072876, by Arch et al., entitled “Platelet derived growth factor b specific antibodies and compositions and uses thereof.” Monomeric anti-PDGF antibodies comprising IgA or IgM constant regions are contemplated in the prior art, however, to the knowledge of the inventor of the present disclosure, dimeric IgA or pentameric IgM antibodies which neutralize PDGF have not previously been disclosed.

In one embodiment, the invention encompasses the use of a dimeric IgA or a pentameric IgM antibody which neutralizes a receptor of platelet derived growth factor (PDGF) in the treatment of ADPKD or other pIgR-associated conditions. In another embodiment, the invention comprises a dimeric IgA antibody or a pentameric IgM antibody which neutralizes a receptor of PDGF. In another embodiment, the invention comprises a dimeric IgA antibody or a pentameric IgM antibody which neutralizes a receptor of PDGF for use in the treatment of ADPKD or other pIgR-associated conditions. For example, the antigen-binding domain of such PDGF receptor-neutralizing antibodies may comprise a binding domain disclosed in United Sates Patent Application Publication Number 20140193402, by Weigand, entitled “Anti PDGFR-beta antibodies and uses thereof”; and in United States Patent Application Publication Number 20090110678, by Ludwig et al., entitled “Receptor antagonists for treatment of metastatic bone cancer.” Monomeric anti-PDGF receptor antibodies comprising IgA or IgM constant regions are contemplated in the prior art, however, to the knowledge of the inventor of the present disclosure, dimeric IgA or pentameric IgM antibodies which neutralize a receptor of PDGF have not previously been disclosed.

MEDICAMENTS AND PREPARATION THEREOF. The antibodies of the invention may be administered in conjunction with pharmaceutically acceptable carriers. Such carriers may comprise compositions which enable the effective storage of active antibody agents and their delivery into the patient, including buffers, preservatives, anti-aggregation agents, and others. In one aspect, the invention is directed to a method of manufacturing of a medicament wherein one or more of the dimeric IgA or pentameric IgM antibodies described herein is combined with formulating compositions to create a solution which is administered to a patient.

Methods of Treatment of ADPKD Utilizing polymeric IgA and IgM Antibodies Against ADPKD-Associated Molecular Targets. In another aspect, the invention comprises methods of treatments utilizing polymeric IgA and IgM antibodies directed to ADPKD-associated molecular targets. In such treatment methods, an animal in need of treatment, for example a human patient suffering from ADPKD, is administered an effective amount of a polymeric IgA or IgM antibody which targets one or more ADPKD-associated molecular targets. In one embodiment, the administered antibodies comprise dimeric IgA or pentameric IgM antibodies. The antibodies of the invention may be administered by any means known in the art. For example intravenous infusion or injection, intramuscular or subcutaneous injection may be utilized.

It will be understood by one of skill in the art that the antibodies of the invention may be administered in a therapeutically effective amount. A therapeutically effective dosage of such antibodies may be determined by one of skill in the art. An exemplary dosage is in the range of 0.1 to 100 mg/kg body weight, for example, in the range of 1-10 mg/kg body weight. Multiple dosages may be administered, for example on a daily, multiple times per week, weekly, or monthly basis to maintain therapeutically effective concentrations at the target tissues.

Other Methods of Treatment. The inventor of the present disclosure has advantageously identified aberrant STAT6 activity as a factor in the overexpression of the pIgR gene in ADPKD cysts. Accordingly, the invention further encompasses the use of polymeric IgA or IgM antibodies to treat any conditions wherein activation of STAT6 is implicated or the upregulation or overexpression of pIgR is present.

In one embodiment, the invention comprises the targeting of polymeric IgA or IgM antibodies to luminal spaces lined by epithelial cells that are not normally accessible to conventional therapeutic antibodies in IgG format. One example is targeting of polymeric IgA or IgM antibodies to the lumens of renal tubules, such antibodies being specific for molecular targets associated with kidney diseases such as chronic kidney disease, diabetic nephropathy, acute kidney injury (AKI) or other conditions leading to chronic kidney failure. Examples of molecular targets present on the luminal side of tubules implicated in these conditions include hepatocyte growth factor and transforming growth factor (TGF)-β and their respective receptors.

In one embodiment, the invention comprises the administration of dimeric IgA or pentameric IgM antibodies in the treatment of renal cysts in other diseases besides ADPKD. Examples of human diseases that are characterized by renal cyst growth include tuberous sclerosis complex, autosomal-recessive polycystic kidney disease, nephronophthisis, Meekel-Gruber syndrome, Bardet-Biedl syndrome, Joubert syndrome, medullary cystic kidney disease, medullary sponge kidney, multicystic dysplastic kidney, Dent's disease, Glomerulocystic kidney disease, Von Hippel-Lindau Syndrome, Acquired Cystic Kidney Disease, acquired simple renal cysts, cystic renal cell carcinoma, cystic nephroma, mixed epithelial and stromal tumor of the kidney, and other ciliopathies. Because renal diseases share many of the underlying molecular mechanisms leading to renal cyst growth, polymeric IgA or IgM antibodies directed against many of the same molecular targets as contemplated for treatment of ADPKD are may be used in the treatment in these other renal cystic diseases as well.

In one embodiment, the invention comprises the administration of dimeric IgA or pentameric IgM antibodies in the treatment of hepatic cysts, wherein such antibodies are specific for molecular targets associated with hepatic cyst onset and/or progression. Examples of molecular targets present on the luminal side of hepatic cysts are IL-8, epithelial neutrophil attractant 78, IL-6, and vascular endothelial growth factor, and their respective receptors.

In another embodiment, the invention comprises the use of dimeric IgA or pentameric IgM antibodies in the treatment of diseases affecting the airways such as asthma, wherein such antibodies are specific for molecular targets associated with factors affecting airway epithelial cells and that are present on the luminal side of the airway epithelium. Examples of asthma-associated molecular targets present on the luminal side of the airway epithelium are basic fibroblast growth factor, transforming Growth Factor-β1, interleukin-6, and their respective receptors.

In one aspect, the antibodies of the invention are human or humanized antibodies directed to human versions of disease-associated molecular targets, and such antibodies may be administered to human patients or test subjects, for example, being administered to a human patient in need of treatment of ADPKD, renal failure, hepatic cysts, or asthma. In another aspect, the antibodies of the invention are comprise non-human IgA, IgM, and J-chain molecules or which are compatible with non-human species and which neutralize non-human molecular targets (e.g. orthologs to the ADPKD molecular targets described herein) and which may be administered to non-human species such as mice, rats, dogs, cats, cattle, pigs, horses, or non-human primates in a veterinary or research context.

All patents, patent applications, and publications cited in this specification are herein incorporated by reference to the same extent as if each independent patent application, or publication was specifically and individually indicated to be incorporated by reference in their entirety. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

Further description of the invention, including exemplary experimental data is provided in the following Example 1.

### EXAMPLE 1

Previous work has indicated that polycystin-1 (PC1), the protein mutated in most cases of ADPKD, can regulate the activity of the transcription factor STAT6, and that aberrant activation of the STAT6 pathway contributes to renal cyst growth (Olsan, et al. (2011) Signal transducer and activator of transcription-6 (STAT6) inhibition suppresses renal cyst growth in polycystic kidney disease. Proceedings of the National Academy of Sciences of the United States of America 108, 18067-18072). STAT6 is typically activated by the cytokines IL4 or IL13 which bind to the heterodimeric IL4/13 receptor (Goenka, S., and Kaplan, M. H. (2011) Transcriptional regulation by STAT6. Immunol Res 50, 87-96). These results indicated that activation of STAT6 in cyst-lining cells causes a positive feedback loop involving the secretion of IL13 into cyst fluid and IL4/IL13 receptor activation at apical membranes of cyst lining epithelial cells. Gene ablation of STAT6—or use of the pharmacological inhibitor leflunomide—lead to reduced renal cyst growth in a PKD mouse model (Olsan, et al. (2011) Signal transducer and activator of transcription-6 (STAT6) inhibition suppresses renal cyst growth in polycystic kidney disease. Proceedings of the National Academy of Sciences of the United States of America 108, 18067-18072).

Due to its non-specificity toward STAT6 and its side-effect profile, leflunomide is unlikely to be useful as a clinical therapy for ADPKD. No small-molecule specific inhibitors of STAT6 are presently available. Highly specific inhibition of signaling pathways can often be achieved by the use of antagonistic antibodies against growth factors or their receptors. For example, antibodies against IL13 or the IL4/13 receptor are currently being tested for asthma therapy. Similarly, antibodies against other growth factors implicated in the pathogenesis of ADPKD, such as EGF and TNF-α, are used for cancer or autoimmune disease therapy (Sliwkowski and Mellman (2013) Antibody therapeutics in cancer. Science 341, 1192-1198; Kopf et al. (2010) Averting inflammation by targeting the cytokine environment. Nat Rev Drug Discov 9, 703-718). Such antagonistic antibodies may potentially be effective for the treatment of ADPKD. Given that highly effective antibodies against numerous promising targets are already available—or under development—as therapeutics for other indications, their may potentially be repurposed them for ADPKD therapy.

Aberrant activation of a targeted signaling pathway in ADPKD is thought to involve involves growth factor/receptor interaction between the cyst fluid and apical plasma membrane of cyst-lining cells. Accordingly, an antagonistic antibody would need to be present in cyst fluid for antibody treatment to be effective. While IgG antibodies are ideal for targets that are accessible via the circulatory system and interstitial fluids it appears unlikely that IgG antibodies would effectively gain access to the lumens of renal cysts in ADPKD. Therefore, the use of IgG antibodies for ADPKD therapy is not promising.

Presented herein is an alternative strategy and exploit the polymeric immunoglobulin receptor (pIgR) to overcome this obstacle. The pIgR is a transmembrane protein that is expressed in many mucosal epithelial cell types. pIgR present at the basolateral plasma membrane can bind to polymeric immunoglobulins (pIg) of the IgA and IgM isotype. Upon binding, the pIgR-pIg complex undergoes transcytosis across the cell to the apical membrane where the extracellular region of pIgR is proteolytically cleaved. This releases the pIg in a complex with the extracellular portion of pIgR, termed secretory component (SC) (Mostov et al. (1995) Regulation of protein traffic in polarized epithelial cells: the polymeric immunoglobulin receptor model. Cold Spring Harb Symp Quant Biol 60, 775-781; Asano et al. (2004) Active synthesis of mouse polymeric immunoglobulin receptor in the epithelial cells of the distal urinary tubule in kidney. Scand J Immunol 60, 267-272). Secretory IgA—the complex of dimeric IgA (dIgA) and SC—is the major antibody isotype in external secretions such as the intestinal lumen, saliva, milk and bile protecting the mucosal environment from infectious agents, such as bacteria, viruses, fungi and parasites (Kaetzel, C. S. (2005) The polymeric immunoglobulin receptor: bridging innate and adaptive immune responses at mucosal surfaces. Immunol Rev 206, 83-99).

Dimeric IgA (dIgA) is composed of two monomeric IgA (mIgA) subunits linked together by disulfide bonds with the so-called J-chain. Since pIgR recognizes the J-chain of dIgA, only dIgA, but not mIgA, can be transcytosed. dIgA is typically produced by plasma cells located in the lamina propria, near the basolateral surface of mucosal epithelia. pIgR has been found in mouse and rat kidney tubule epithelial cells , and pIgR expression can be regulated by water deprivation, vasopressin administration or renal ischemia-reperfusion in rats. dIgA can be found in urine, suggesting that it can reach the urinary space by pIgR-mediated transcytosis (Rice et al. (1998) Regulation of the polymeric immunoglobulin receptor by water intake and vasopressin in the rat kidney. Am J Physiol 274, F966-977.

Rice et al. (1999) Expression of the polymeric immunoglobulin receptor and excretion of secretory IgA in the postischemic kidney. Am J Physiol 276, F666-673). pIgR expression has been shown to be regulated by IL-4, TNF-α and IFN-γ in airway, intestinal and mammary gland epithelial cells (Loman et al. (1999) Interleukin-4 and interferon-gamma synergistically increase secretory component gene expression, but are additive in stimulating secretory immunoglobulin A release by Calu-3 airway epithelial cells. Immunology 96, 537-543), and a STAT6 binding domain has been identified in intron 1 of the pIgR gene (Schjerven, H., Brandtzaeg, P., and Johansen, F. E. (2000) Mechanism of IL-4-mediated up-regulation of the polymeric Ig receptor: role of STAT6 in cell type-specific delayed transcriptional response. J Immunol 165, 3898-3906; Johansen and Brandtzaeg, (2004) Transcriptional regulation of the mucosal IgA system. Trends Immunol 25, 150-157). Altogether, these data suggest that the kidney can use the dIgA/pIgR system and that it can be upregulated to protect the urinary space against pathogens.

It was previously found that STAT6 is activated in cyst-lining cells in PKD, so it was hypothesized that this may lead to increased pIgR expression and that pIgR could be exploited to transport dIgA across the epithelium into the cyst lumens. Here is shown that pIgR is indeed highly expressed in renal cysts and is processed into SC indicative of active transcytosis. Consequently, murine and human cyst fluids contain high levels of dIgA. When dIgA is injected into mice, it accumulates preferentially in polycystic kidneys compared to normal kidneys. In contrast, very little injected IgG accumulates in polycystic kidneys. These results indicate that therapeutic antibodies can be targeted by pIgR-mediated transcytosis to the lumens of renal cysts if they are in the dIgA format. Since renal cysts are enclosed spaces, dIgA antibodies are expected to accumulate in their lumens over time while they are rapidly being eliminated from other secretions and the circulation. Therefore, pIgR-mediated targeting of dIgA antibodies may be used in ADPKD therapy with high specificity towards the target organ.

### Experimental Procedures

### Animals

The Institutional Animal Care and Use Committee of the University of California at Santa Barbara approved all animal experiments. Pkd1^{cond/cond}, Pkd1^{cond/cond}:Nestin^{cre}, and wt/bpk colonies were maintained under standard vivarium conditions. STAT6^{-/-} animals on BALB/c background were obtained from The Jackson Laboratory (Bar Harbor, Me.) and crossed with the wt/bpk animals as previously described.

### Antibodies

Goat anti-mouse pIgR antibody was obtained from R&D Systems, Inc. (Minneapolis, Minn.). Rabbit anti-human IgA and mouse anti-β-actin antibody were from Sigma-Aldrich Co. LLC. (Saint Louis, Mo.). Mouse anti-pIgR (C-terminus; SC166) and guinea pig anti-SC were kindly provided by Keith Mostov (UCSF) (25). HRP- and fluorescence-conjugated secondary antibodies were obtained from Jackson ImmunoResearch Laboratories, Inc. (West Grove, Pa.) and Santa Cruz Biotechnology, Inc. (Dallas, Tex.). Rhodamine-conjugated Dolichos biflorus agglutinin (DBA) was from Vector Laboratories, Inc (Burlingame, Calif.).

### Human Samples

Normal and ADPKD kidney samples were obtained through the National Disease Research Interchange (NDRI).

In vivo Immunoglobulin Injection
10 μg of biotinylated mouse IgA or biotinylated mouse IgG (BD Biosciences, San Jose, Calif.) were injected i.p. into wild-type or bpk/bpk mice at postnatal day 21. Animals were euthanized 24 hours post injection. 5 μm sections from formalin-fixed paraffin-embedded kidney tissue were deparaffinized in xylene, then rehydrated through a series of alcohol, followed by antigen retrieval using 4×5 min microwave sessions in 10 mM trisodium citrate, pH 6.0. Sections were blocked with 1% BSA in Tris-buffered saline with 0.1% Tween-20, followed by blocking of endogenous peroxidase activity using 3% H₂O₂ in Tris-buffered saline. Sections were incubated with ABC Reagent from Elite Kit (Vector Labs) followed by application of DAB.

100 μg of purified human IgA or human IgG (Sigma-Aldrich Co. LLC. Saint Louis, Mo.) were injected i.p. into wild-type or bpk/bpk mice at postnatal day 18-19, or into Pkd1^{cond/cond}:Nestin^{cre} mice at 6 months of age. Animals were euthanized 12 or 24 hours post injection and kidney tissues were either flash-frozen in liquid nitrogen, or cyst fluids were aspirated with a fine needle. Flash-frozen kidneys and cyst fluids were lysed in SDS-buffer buffer. Samples were separated by non-reducing 6% SDS-PAGE or 4-15% gradient SDS-PAGE gels, transferred to nitrocellulose and subjected to immunoblot using anti-human IgA, anti-human IgG or an anti-human kappa light chain HRP conjugate (Life Technologies, Thermo Fisher Scientific, Inc., Waltham, Mass.). Western blots were quantified by film densitometry. The amount of human IgA retained in both kidneys was calculated and represented as the percentage of original injected material.

qPCR
Total RNA was isolated from mouse inner-medullary collecting duct (IMCD3) cells treated for 18 hours with DPBS (Mediatech, Inc. A Corning Subsidiary, Manassas, Va.), 100 ng/mL mouse IL4 (R&D Systems, Inc., Minneapolis, Minn.) or 100 ng/mL mouse IL13 (Cell Signaling Technology, Inc., Danvers, Mass.) using the RNeasy Plus Mini Kit (Qiagen, Inc., Valencia, Calif.). RNA (2 μg) was converted to cDNA using M-MLV Reverse Transcriptase (Promega Corp., Madison, Wis.). Primers used for qPCR amplification for pIgR were as described in Asano et al. (2004) Active synthesis of mouse polymeric immunoglobulin receptor in the epithelial cells of the distal urinary tubule in kidney. Scand J Immunol 60, 267-272; primers for cross-species β-actin, were as described in Barlucchi et al. (2001) Canine ventricular myocytes possess a renin-angiotensin system that is upregulated with heart failure. Circ Res 88, 298-304. Amplification program included annealing temperature of 55° C. and used GoTaq qPCR Master Mix (Promega Corp., Madison, Wis.) and the Stratagene Mx3000P qPCR System (Agilent Technologies, Inc., Santa Clara, Calif.). Data analysis and statistics using GraphPad Prism (GraphPad Software, Inc., La Jolla, Calif.).

### Results

pIgR is Expressed in Renal Cyst-lining Cells and Processed into SC
Previously, it was found that STAT6 is aberrantly activated in renal cyst-lining epithelial cells in two mouse models of PKD, the Bpk model and the human-orthologous Pkd1^{cond/cond}:Nestin^{cre} model. In addition, significant amounts of the STAT6 activating cytokine IL13 are present in cyst fluid in these models. It was tested whether STAT6 activation may lead to increased pIgR expression in these models. Immunoblotting revealed that pIgR expression is increased in Bpk polycystic kidneys compared to kidneys from age-matched control animals. In addition, a significant fraction of pIgR is processed leading to the SC cleavage product. This indicates that a significant fraction of pIgR must have transcytosed polymeric immunoglobulins across renal epithelial cells, and that the resulting SC fragment is unable to be excreted into the urinary space, suggesting that it is trapped in cyst lumens. To test whether increased pIgR expression may be due to aberrant STAT6 activation, mice lacking STAT6 were tested. pIgR expression is strongly reduced—but not completely eliminated—in kidneys of bpk/bpk:STAT6^{-/-} mice compared to bpk/bpk mice. This indicates that STAT6 is not necessary for a basal level of pIgR expression but is responsible for the observed increased expression in bpk/bpk mice. Similarly, pIgR is expressed and processed into SC in the human orthologous Pkd1^{cond/cond}:Nestin^{cre} mouse model compared to control Pkd1^{cond/cond} mice.

Immunofluorescence microscopy revealed that pIgR expression in polycystic kidneys of bpk/bpk mice is confined to epithelial cells. Virtually all cysts exhibit at least a basal expression level with particularly intense staining in numerous smaller cysts. pIgR is expressed both in cysts that stain positive or negative for the collecting duct marker Dolichos biflorus agglutinin (DBA). In control kidneys, pIgR expression is low or absent in most tubules except for occasional cells in DBA-negative tubules, consistent with previous findings.

Treatment of mouse inner-medullary collecting duct cells (IMCD) in vitro with IL4 or IL13 to activate STAT6 significantly increases the mRNA expression of pIgR. While the results of IL4 treatment are consistent with previous findings in intestinal epithelial cells, importantly, IL13 treatment has a similar or greater effect on pIgR expression, which has not been previously demonstrated. Altogether, these results indicate that STAT6 regulates the expression of pIgR in renal epithelial cells and that pIgR expression is increased in cyst-lining cells. Furthermore, the results suggest that pIgR actively undergoes transcytosis in polycystic kidneys leading to the accumulation of the SC fragment.

pIgR and SC are Highly Expressed in Human ADPKD Kidneys
Next, it was determined if the increased pIgR expression and accumulation of SC observed in mouse models of PKD also occurs in human ADPKD kidneys. It was observed that pIgR expression is strongly increased in ADPKD kidneys compared to normal human kidneys. Additionally, strong signals for the SC fragment of pIgR are detected in aspirated cyst fluids from ADPKD kidneys. Since pIgR undergoes transcytosis and cleavage at the apical plasma membrane when it is bound to polymeric IgA or IgM, this result indicates that pIgR must actively transport these secretory immunoglobulins across cyst-lining epithelial cells into cyst fluid. Immunofluorescence microscopy showed little to no detectable pIgR expression in normal human kidney. This is consistent with previous findings from normal human kidneys (Abramowsky and Swinehart (1986) Secretory immune responses in human kidneys. Am J Pathol 125, 571-577). In contrast, there is strong pIgR immuno-staining in epithelial cells lining most cysts in ADPKD kidneys.
Endogenous dIgA Accumulates in Renal Cyst Fluids
IgA in normal serum is primarily in the monomeric form but a fraction is dimeric which, in mice, is largely cleared by transport into bile (Monteiro, R. C. (2010) Role of IgA and IgA fc receptors in inflammation. J Clin Immunol 30, 1-9). To directly determine whether the observed high expression level of pIgR and its processing into SC leads to transport of dIgA into renal cyst fluids, total kidney lysates were examined from wild-type and cystic mice by immunoblot analysis. Under non-reducing conditions, dIgA was partially preserved during electrophoresis and was visible as a ˜250 kDa band in contrast to mIgA at ˜130 kDa. The amount of dIgA was strongly increased in kidneys from cystic Pkd1^{cond/cond}:Nestin^{cre} mice in comparison to normal Pkd1^{cond/cond} mice. The relative amount of dIgA to mIgA in cystic kidneys was also increased in comparison to serum, consistent with the view that dIgA actively accumulates in cystic kidneys by pIgR-mediated transport. When examining aspirated cyst fluid from Pkd1^{cond/cond}:Nestin^{cre} mice, strong bands for dIgA were observed, suggesting that dIgA indeed undergoes pIgR-mediated transport across the cyst-lining epithelium and accumulates in cyst fluids.
Parenterally Administered dIgA is Targeted to Renal Cysts more Effectively than IgG
To determine whether exogenous dIgA can be effectively delivered to renal cysts, biotinylated mouse IgA (a mixture of mIgA and dIgA) or biotinylated mouse IgG were administered by i.p. injection into bpk/bpk mice. 24-hours post injection the localization of biotinylated immunoglobulins was analyzed by immunohistochemistry. No biotin signals were detected in the kidneys of uninjected mice or mice injected with biotinylated-IgG. In contrast, cyst lining epithelial cells stained positive in mice injected with biotinylated-IgA, suggesting the exogenous IgA has been endocytosed in these cells and may be undergoing transcytosis.

To directly observe the in vivo transport and renal accumulation of exogenous, unmodified immunoglobulins human dIgA (or human IgG) were administered into bpk/bpk mice or age-matched wild-type mice. The dIgA/pIgR interaction is highly conserved among mammalian species. Consequently, human dIgA, when injected into rodents, is recognized and transcytosed normally by rodent pIgR in vivo (Giffroy et al. (1998) In vivo stimulation of polymeric Ig receptor transcytosis by circulating polymeric IgA in rat liver. International immunology 10, 347-354). The use of human-specific antibodies against IgA (or IgG) then enables the detection of injected human immunoglobulins over the large background of endogenous murine immunoglobulins. 12-hours post i.p. injection of human IgA (a mixture of mIgA and dIgA) or human IgG, kidney lysates were analyzed by immunoblotting. It was observed that dIgA accumulated preferentially in kidneys of bpk/bpk cystic mice compared to wild-type controls. In contrast, very little—if any—injected human IgG was detectable in kidneys of either cystic or control mice. Altogether, these results suggest that IgG antibodies do not effectively target to polycystic kidneys but that dIgA antibodies undergo pIgR-mediated transport into the lumen of renal cysts where they accumulate.

It was hypothesized that the human dIgA that was found retained in kidneys of wild-type mice 12 hours after i.p. injection would eventually be cleared by urinary excretion whereas human dIgA retained in polycystic kidneys should be retained long-term because it will be trapped inside cysts. To test this hypothesis the above experiment was repeated, but kidneys were analyzed 24 hours post injection of human dIgA. At this time point, very little injected dIgA is still retained in wild-type kidneys. In contrast, approximately 7% of the injected human dIgA was recovered from polycystic kidneys. Based on the kidney weight and the fact that kidneys in this model consist of ˜50% cyst fluid, it was estimated that the concentration of parenterally administered dIgA reached 7 μg/ml in cyst fluid after 24 hours. This assumes that the retained dIgA in polycystic kidneys of Bpk mice is primarily present in cyst fluid may be an over-estimate. However, given that the 50%-effective concentration of currently used therapeutic IgG antibodies is typically in the range of 1-200 ng/ml or less, this indicates that therapeutically effective concentrations of dIgA antibodies are achievable in renal cyst fluid.

To directly test whether injected dIgA is transported into cyst lumens, human dIgA was injected into either 16 day old bpk/bpk mice or into 6 month old Pkd1^{cond/cond}:Nestin^{cre} mice. Cyst fluids were aspirated and analyzed by immunoblotting. Injected, human dIgA was from cyst fluids from both mouse models after 24 hours indicating that parenterally administered dIgA is taken up by cyst lining cells and transcytosed into the lumen.

Altogether, these results indicate that dIgA efficiently accumulates in polycystic kidneys where it persists for extended periods of time.

### Discussion

Herein is disclosed a novel strategy to target therapeutic or diagnostic antibodies to polycystic kidneys, and in particular to the lumens of renal cysts. While virtually all immunoglobulins designed for clinical use are in IgG format, these antibodies are not expected to gain access to the luminal space in polycystic kidneys. Therefore, such antibodies will likely be ineffective if their targets are present on the apical surface of cyst-lining cells or within the cyst fluid. Several growth factors have previously been identified in renal cyst fluid and implicated in driving cyst growth such as EGF, HGF, and TNF-α. Other previous results indicated that STAT6 is aberrantly activated in cyst-lining cells due to auto/paracrine stimulation of the IL4/13 receptor by IL13 present in cyst fluid. Inhibition of these pathways using antagonistic antibodies requires that the antibodies gain access to the cystic space.

Using a combination of PKD mouse models and human ADPKD tissues, it was shown that renal cyst-lining epithelial cells express the pIgR, and that this results in active transport of dIgA from the circulation across the epithelium where secretory IgA accumulates in the cyst fluids. Data suggested that aberrant STAT6 activation contributes to the observed high level of pIgR expression.

Altogether, the results presented herein demonstrate that the pIgR-mediated transport of dIgA into renal cyst lumens can be exploited to target therapeutic antibodies to this compartment. Besides the ability to initially target dIgA antibodies to renal cysts, an additional benefit would be that the dIgA antibodies will remain and accumulate in cyst fluids because renal cysts lack a connection to the tubular system. This is in contrast to virtually all other epithelial tissues that express the pIgR and transport dIgA to external secretions which are lost over time. For example, in mice the bulk of dIgA present in plasma is cleared via transcytosis into bile. Similarly, in humans dIgA is excreted via the intestinal epithelium, salivary glands, and lungs. Therefore, parenteral administration of dIgA is not expected to lead to accumulation in tissues that normally express the pIgR, which should limit off-target side effects. Furthermore, IgA and SC have been found in hepatic cyst fluid from ADPKD patients (Everson et al. (1990) Functional similarities of hepatic cystic and biliary epithelium: studies of fluid constituents and in vivo secretion in response to secretin. Hepatology 11, 557-565), indicating that therapeutic dIgA antibodies may also effectively target to liver cysts. Given that secretory IgA is highly stable and can withstand extreme environments (Davidson, L. A., and Lonnerdal, B. (1987) Persistence of human milk proteins in the breast-fed infant. Acta Paediatr Scand 76, 733-740), it is likely that it exhibits a long half-life in renal cyst fluid which may allow low-frequency dosing similar to established therapies with IgG antibodies.

While there are currently no approved antibodies using isotypes other than IgG, the idea of using IgA antibodies for cancer treatment has recently been investigated (Boross et al. (2013) IgA EGFR antibodies mediate tumour killing in vivo. EMBO Mol Med 5, 1213-1226; Lohse et al. (2012) Characterization of a mutated IgA2 antibody of the m(1) allotype against the epidermal growth factor receptor for the recruitment of monocytes and macrophages. J Biol Chem 287, 25139-25150; and Lohse, et al. (2011) Recombinant dimeric IgA antibodies against the epidermal growth factor receptor mediate effective tumor cell killing. J Immunol 186, 3770-3778). Beyond treatment for ADPKD, dIgA antibodies are useful for treatment of other disorders in which pIgR-mediated antibody targeting to epithelial luminal spaces would be desirable. This includes other renal disorders such as chronic kidney disease, lung diseases such as asthma or cystic fibrosis etc. In an alternative implementation of the concepts disclosed and demonstrated herein, pIgR-binding peptides that can be linked to payloads may be used for treating tissues, structures, and pathologies wherein high pIgR expression is observed, as has been investigated for targeting other mucosal epithelia (White and Capra (2002) Targeting mucosal sites by polymeric immunoglobulin receptor-directed peptides. J Exp Med 196, 551-555; Braathen et al. (2006) Identification of a polymeric Ig receptor binding phage-displayed peptide that exploits epithelial transcytosis without dimeric IgA competition. J Biol Chem 281, 7075-7081).

Further data and description of the experiments summarized in Example 1 is found in Olsan et al., Exploitation of the Polymeric Immunoglobulin Receptor for Antibody Targeting to Renal Cyst Lumens in Polycystic Kidney Disease, Journal of Biological Chemistry (2015): jbc-M114.

What is claimed is:

1-111. (canceled)

112. A method of treating a patient suffering from autosomal-dominant polycystic kidney disease; comprising
the administration of a therapeutically effective dosage of a therapeutic antibody,
wherein the therapeutic antibody comprises a dimeric IgA or a pentameric IgM antibody;
wherein the therapeutic antibody will neutralize a growth factor associated with the autosomal-dominant polycystic kidney disease state, or a receptor thereof; and
wherein pIgR-mediated transcytosis enables delivery of the therapeutic antibody to the lumen of renal cysts.

113. The method of claim 112, wherein
the administered antibody is a dimeric IgA antibody.

114. The method of claim 112, wherein
the administered antibody is a pentameric IgM antibody.

115. The method of claim 112, wherein
the antigen-binding region of the administered antibody specifically binds to interleukin-13 or a receptor thereof.

116. The method of claim 112, wherein
the antigen-binding region of the administered antibody specifically binds to interleukin-4 or a receptor thereof.

117. The method of claim 112, wherein
the antigen-binding region of the administered antibody specifically binds to epidermal growth factor or a receptor thereof.

118. The method of claim 112, wherein
the antigen-binding region of the administered antibody specifically binds to hepatocyte growth factor or a receptor thereof.

119. The method of claim 112, wherein
the antigen-binding region of the administered antibody specifically binds to transforming growth factor alpha or a receptor thereof.

120. The method of claim 112, wherein
the antigen-binding region of the administered antibody specifically binds to transforming growth factor beta or a receptor thereof.

121. The method of claim 112, wherein
the antigen-binding region of the administered antibody specifically binds to tumor necrosis factor alpha or a receptor thereof.

122. The method of claim 112, wherein
the antigen-binding region of the administered antibody specifically binds to interleukin 6 or a receptor thereof.

123. The method of claim 112, wherein
the antigen-binding region of the administered antibody specifically binds to HER2 or a receptor thereof.

124. The method of claim 112, wherein
the antigen-binding region of the administered antibody specifically binds to platelet-derived growth factor or a receptor thereof.

125. An antibody, comprising
a dimeric IgA or a pentameric IgM antibody; and
an antigen-binding region which specifically binds epidermal growth factor receptor.
